(19) United States
(12) Patent Application Publication
(10) Pub. No.: US 2026/0034171 A1
(43) Pub. Date: Feb. 5, 2026

(54) Composition for Improving Cognitive Function

(71) Applicant: Stephen C. Perry, Norwood, MA (US)

(72) Inventor: Stephen C. Perry, Norwood, MA (US)

(21) Appl. No.: 19/285,154

(22) Filed: Jul. 30, 2025

Related U.S. Application Data
(60) Provisional application No. 63/677,159, filed on Jul. 30, 2024.

(51) Int. Cl. A61K 33/06; A61K 31/05; A61K 31/192; A61K 31/197; A61K 31/201; A61K 31/4418; A61K 31/4525; A61K 31/55; A61K 31/685; A61K 36/81

(52) U.S. Cl. CPC A61K 33/06; A61K 31/05; A61K 31/192; A61K 31/197; A61K 31/201; A61K 31/4418; A61K 31/4525; A61K 31/55; A61K 31/685; A61K 36/81

(57) ABSTRACT

Compositions and methods are described for improving brain health and cognitive function and for preventing and treating Alzheimer's disease and other age-related dementia diseases and conditions for intake as a dietary or nutritional supplement, for example, as a powdered drink mix. Compositions for providing an energy source to the brain during exercise are also described. The composition includes a source of choline, a source of magnesium, at least one taurine-containing composition, a source of ashwagandha root, and phosphatidylserine. The source of choline can be alpha-glycerylphosphorylcholine or citicoline and the source of magnesium can be magnesium-L-threonate or magnesium glycinate. The composition can further include at least one of oleic acid, oleanolic acid, ursolic acid, curcumin, piperine, galantamine, huperzine-A, a source of vitamin B5, L-tyrosine, and beta-hydroxybutyrate.

### CROSS-REFERENCE TO RELATED APPLICATIONS

This application is a nonprovisional application of and claims priority from U.S. provisional patent application Ser. No. 63/677,159 filed on Jul. 30, 2025. The foregoing application and U.S. Pat. No. 10,881,125 are incorporated in their entirety herein by reference.

### FIELD OF THE INVENTION

The invention relates to a brain health and disease prevention and treatment. More particularly, the invention relates to compositions and methods for improving cognitive function and for the prevention and treatment of Alzheimer's disease and other age-related dementia diseases and conditions.

### BACKGROUND

Alzheimer's disease is a debilitating degenerative affliction of the nervous system, which will become increasingly common during the next three decades as the American population ages. In the baby-boomer generation in particular, has aged. Current estimates reveal that by 2035, when the average individual of the “baby boomer” generation is age 85, potentially 50% of Americans will have developed Alzheimer's disease. By providing a treatment that can delay the onset of symptoms of Alzheimer's by as little as five years, at age 85, fifty percent of the United States cases of Alzheimer's could be eliminated. Alzheimer's disease has a negative impact on an individual's memory and cognitive functions, ability to perform the simple activities of daily living, and causes behavioral problems among its sufferers with which families must learn to cope. Typically, Alzheimer's disease also reduces the lifespan of an individual by increasing one's risk of succumbing to secondary infections and illnesses. The disease is associated with the accumulation of β-amyloid plaques in the brain which leads to the eventual destruction of brain cells. The primary cause of Alzheimer's disease may be flaws in the metabolic processes governing production, accumulation, or disposal of the β-amyloid protein fragments. Therefore, treatments for Alzheimer's disease often have focused on dissolving β-amyloid or preventing the aggregation of the β-amyloid fragments into plaque formations.

Research has shown that people indigenous to the Indian subcontinent exhibit a much lower incidence of Alzheimer's disease than people living in the United States. Moreover, the research indicated that less than one percent of individuals in the examined population on the Indian subcontinent developed Alzheimer's and that the overall incidence rates of the disease in that location are among the lowest ever reported. Some researchers have attributed the low incidence of Alzheimer's disease in India to regional diets that are high in the curry spice, turmeric. Turmeric contains a substance, curcumin, which has demonstrated metabolic activity similar to non-steroidal anti-inflammatory drugs.

Curcumin, a phenolic antioxidant phytochemical derived from the turmeric plant (Curcuma longa), is an effective antioxidant, antispasmodic, anti-inflammatory, anticoagulant, anticarcinogenic, and aids immunomodulatory activities and wound healing in the body. Curcumin exhibits an affinity for β-amyloid and both inhibits the aggregation of β-amyloid fragments into plaque formations as well as dissolves existing β-amyloid plaques.

As an antioxidant, curcumin removes harmful free radicals from the body, thereby protecting the human body, and particularly, the brain, by preventing lipid peroxidation. This antioxidant property of curcumin limits the formation and accumulation of β-amyloid plaques within the brain. The non-steroidal anti-inflammatory activity of curcumin includes inhibiting cyclooxygenase 2, nuclear factor KB-mediated transcription of inflammatory cytokines, and the inhibition of inducible nitric oxide synthase. Research has shown that parenteral administration of both low and high doses of curcumin reduces inflammation in the brain. Curcumin may also stimulate microglial phagocytosis of amyloid in the brain as well as destroy plaques that accumulate within the brain.

One disadvantage to the use of curcumin as an oral dietary supplement is that curcumin is poorly absorbed from inside the gastrointestinal tract into the human bloodstream. When ingested, curcumin normally remains in the gastrointestinal tract and uptake into the bloodstream is negligible. Even with high dietary intake, curcumin is rapidly glucuronidated after ingestion, thereby resulting in low plasma levels of curcumin. Thus, current research indicates that orally administered curcumin, for the most part, is excreted in the feces without being absorbed and processed by the human body. To make available the beneficial effects of curcumin to Alzheimer's sufferers, the bioavailability of curcumin in the human body must be increased.

Piperine, a botanical extract from the fruits of Piper nigrum (black pepper) and Piper longum, has been determined to increase the bioavailability of curcumin in the body when both curcumin and piperine are taken together. Studies have shown that oral administration of 20.0 mg of piperine with 2.0 grams of curcumin increases the bioavailability of curcumin by 2,000%.

Galantamine, a natural plant extract derived from the daffodil, snowdrop, and the spider lily, is an acetylcholinesterase inhibitor. Acetylcholinesterase is an enzyme that breaks down acetylcholine in the synaptic cleft between nerve cells. Acetylcholine is involved in memory and learning processes. Research has shown that the level of acetylcholine present in the nervous system of Alzheimer's patients is abnormally low. Galantamine increases the level of acetylcholine present in the brain by inhibiting the activity of acetylcholinesterase. In addition to being a useful therapeutic agent in the treatment of Alzheimer's disease, galantamine may also be useful for treating mild cognitive impairment, an age-related condition often diagnosed as a precursor condition and risk factor for developing Alzheimer's. Galantamine is also effective in treating Alzheimer's disease because it works to modulate nicotinic receptors on brain cells, which respond to acetylcholine, thereby preserving the number and functional integrity of nicotinic receptors in the brain. In Alzheimer's patients, the number and functional integrity of nicotinic receptors is diminished resulting in fewer receptors for acetylcholine. The benefits of treating Alzheimer's with galantamine are not affected by previous treatments using other acetylcholinesterase inhibitors. Thus, galantamine may work to effectively halt the progression of Alzheimer's disease and also allows a patient to regain some memory and cognitive functions as well as being able to perform some simple tasks of daily living.

Huperzine A, an alkaloid plant extract from the club moss Huperzia serrata, is a nootropic agent that also strongly inhibits the activity of acetylcholinesterase. As huperzine A inhibits the break down of acetylcholine by acetylcholinesterase, more acetylcholine becomes available to stimulate neurons. Huperzine A provides a long-lasting, potent means for inhibiting the enzymatic activity of acetylcholinesterase, thereby increasing the amount of acetylcholine present within the nervous system.

Oleic acid increases the absorptivity of the intestines so that food and nutrients can be fully absorbed into the body. In this way, oleic acid aids the body in digesting and processing substances that are difficult to digest. Bile (phosphatidylcholine) is the body's principle source of oleic acid in the small intestine, however only small amounts are produced. Oleanolic acid and ursolic acid share similar properties with oleic acid and also enhance the effects of curcumin in the body.

Vitamin B5, or pantothenic acid, is used by the human body along with choline to form acetylcholine. Thus, as important precursors to the formation of acetylcholine, which is often present in reduced levels in Alzheimer's patients, choline and vitamin B5 are necessary nutritional supplements for preventing the onset of symptoms associated with neurodegenerative disorders, such as Alzheimer's.

A need exists for compositions and methods for preventing and treating the symptoms of Alzheimer's disease and other age-related dementia diseases and conditions.

A need also exists for compositions and methods for improving cognitive function and treating the symptoms of traumatic brain injury.

A need also exists for compositions and methods for providing a source of energy for the brain during exercise.

A need also exists for compositions for preventing and treating the symptoms of Alzheimer's disease and other age-related dementia diseases and conditions in which the bioavailability of active ingredients is increased.

### SUMMARY

The invention relates to compositions and methods for improving brain health and cognitive function. The invention also relates to compositions and methods for preventing and treating Alzheimer's disease and other age-related dementia diseases and conditions. The invention also relates to compositions and methods for improving cognitive function and treating traumatic brain injuries. One embodiment of the composition includes a source of choline, a source of magnesium, at least one taurine-containing composition, a source of ashwagandha root, and phosphatidylserine. Another embodiment of the composition includes alpha-glycerylphosphorylcholine, magnesium glycinate, at least one taurine-containing composition, a source of ashwagandha root, phosphatidylserine, a source of turmeric root, piperine, oleic acid, oleanolic acid, ursolic acid, huperzine A, and vitamin B5. Yet another embodiment of the composition includes alpha-glycerylphosphorylcholine, magnesium glycinate, at least one taurine-containing composition, phosphatidylserine, a source of turmeric root, and piperine. Still another embodiment of the composition includes magnesium glycinate, at least one taurine-containing composition, a source of ashwagandha root, phosphatidylserine, quercetin dihydrate, a source of turmeric root, alpha-lipoic acid, nicinamide, piperine, astaxanthin, glutamine, and D-ribose.

Various embodiments of the composition that are useful for particular purposes may also include oleic acid, oleanolic acid, ursolic acid, curcumin, piperine, galantamine, huperzine-A, vitamin B5, L-tyrosine, beta hydroxybutyrate, and other ingredients.

Consumption of the composition as a dietary or nutritional supplement provides an advantage to health in humans by preventing and treating the symptoms of Alzheimer's disease and other age-related dementia diseases and conditions. Consumption of the composition as a dietary or nutritional supplement also provides an advantage to health in humans by improving cognitive function for people generally. Consumption of the composition as a dietary or nutritional supplement provides an advantage to health in humans by treating the symptoms of and improving cognitive function in persons with traumatic brain injuries.

The compositions and methods described herein also provide an advantage to young adults and to persons engaged in exercise by providing a source of energy for the brain.

The compositions and methods described herein provide another advantage in preventing and treating the symptoms of Alzheimer's disease and other age-related dementia diseases and conditions by increasing the bioavailability of active ingredients by increasing their absorption during digestion.

Accordingly, the invention features a composition for improving cognitive function and for preventing and treating symptoms of Alzheimer's disease and dementia. The composition includes: a source of choline, a source of magnesium, at least one taurine-containing composition, a source of ashwagandha root, and phosphatidylserine.

In another aspect, the invention can feature the source of choline being or including alpha-glycerylphosphorylcholine or citicoline.

In another aspect, the invention can feature the source of magnesium being or including magnesium L-threonate or magnesium glycinate.

In another aspect, the invention can feature the composition further including a source of vitamin B5 that is or includes D-pantothenic acid, calcium pantothenate, sodium pantothenate, pantethine, or a combination of two or more of the foregoing.

In another aspect, the invention can feature the composition further including: a source of turmeric root, piperine, uridine monophosphate, L-theanine, and vitamin B5.

In another aspect, the invention can feature the composition further including: a source of turmeric root, piperine, oleic acid, oleanolic acid, ursolic acid, huperzine A, and vitamin B5.

In another aspect, the invention can feature the composition further including a source of turmeric root and piperine.

In another aspect, the invention can feature the composition further including: a source of turmeric root, piperine, oleic acid, oleanolic acid, ursolic acid, huperzine A, L-tyrosine, beta-hydroxybutyrate, and vitamin B5.

In another aspect, the invention can feature the composition further including: quercetin dihydrate, a source of turmeric root, alpha-lipoic acid, nicinamide, piperine, astaxanthin, glutamine, and D-ribose.

In another aspect, the invention can feature the composition further including: piperine, curcumin, L-theanine, ursolic acid, acetyl-L-carnitine, and vitamin B5.

In another aspect, the invention can feature the composition including: about 250 to about 1,000 mg of citicoline, or about 300 to about 600 mg of alpha-glycerylphosphorylcholine; about 1,000 to about 2,000 mg of magnesium L-threonate, or about 100 to about 300 mg of magnesium glycinate; about 300 to about 600 mg of a source of ashwagandha root; about 100 to about 300 mg of phosphatidylserine; about 5 to about 15 mg of piperine; about 250 to about 1,000 mg of at least one taurine-containing composition; about 250 to about 700 mg of curcumin; about 100 to about 200 mg of L-theanine; about 50 to about 200 mg of ursolic acid; about 1,500 to about 3,000 mg of acetyl-L-carnitine; and about 250 to about 1,000 mg of vitamin B5.

In another aspect, the invention can feature the source of choline being or including alpha-glycerylphosphorylcholine, citicoline, phosphatidylcholine, phosphocholine, free choline, or a combination of two or more of the foregoing.

In another aspect, the invention can feature the source of magnesium being or including magnesium glycinate, magnesium L-threonate, magnesium citrate, magnesium oxide, magnesium chloride, magnesium malate, magnesium sulfate, magnesium taurate, magnesium lactate, or a combination of two or more of the foregoing.

In another aspect, the invention can feature the at least one taurine-containing composition being or including taurine, taurine glycinate, taurine oleate, taurine ethanoleate, taurine ethanolamine, a taurine ester, a taurine salt, a taurine amino alcohol, taurocholic acid, a taurolipid, a lipophilic taurine analog, N-acetyltaurine, N-propionyltaurine, a taurine-fatty acid conjugate, a taurine amide derivative, or a combination of two or more of the foregoing.

In another aspect, the invention can feature the composition further including at least one of the following additional ingredients: citric acid; a flavoring agent; gum acacia; a composition for masking or reducing bitter flavors; a sweetener, and bamboo extract.

The invention also features a composition for preventing and treating symptoms of Alzheimer's disease and dementia. The composition includes: alpha-glycerylphosphorylcholine, magnesium glycinate, at least one taurine-containing composition, a source of ashwagandha root, phosphatidylserine, a source of turmeric root, piperine, oleic acid, oleanolic acid, ursolic acid, huperzine A, and vitamin B5.

In another aspect, the invention can feature the composition further including L-tyrosine, beta-hydroxybutyrate, or both.

In another aspect, the invention can feature the composition further including at least one of the following additional ingredients: citric acid; a flavoring agent; gum acacia; a composition for masking or reducing bitter flavors; a sweetener, and bamboo extract.

The invention also features a composition for preventing and treating symptoms of Alzheimer's disease and dementia. The composition includes: alpha-glycerylphosphorylcholine, magnesium glycinate, at least one taurine-containing composition, phosphatidylserine, a source of turmeric root, and piperine.

The invention also features a composition for improving cognitive function and treating symptoms of traumatic brain injury. The composition includes: magnesium glycinate, at least one taurine-containing composition, a source of ashwagandha root, phosphatidylserine, quercetin dihydrate, a source of turmeric root, alpha-lipoic acid, nicinamide, piperine, astaxanthin, glutamine, and D-ribose.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents and other references mentioned herein are incorporated by reference in their entirety. In the case of conflict, the present specification, including definitions will control.

### DETAILED DESCRIPTION

The present invention is best understood by reference to the detailed description set forth herein. Embodiments of the invention are discussed below; however, those skilled in the art will readily appreciate that the detailed description given herein is for explanatory purposes as the invention extends beyond these limited embodiments. For example, in light of the teachings of the present invention, those skilled in the art will recognize a multiplicity of alternate and suitable approaches, depending upon the needs of the particular application, to implement the functionality of any given detail described herein beyond the particular implementation choices in the following embodiments described and shown. That is, numerous modifications and variations of the invention may exist that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

The present invention should not be limited to the particular methodology, compounds, materials, manufacturing techniques, uses, and applications, described herein, as these may vary. The terminology used herein is used for the purpose of describing particular embodiments only, and is not intended to limit the scope of the present invention. As used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” may be a reference to one or more steps or means and may include sub-steps and subservient means.

All conjunctions used herein are to be understood in the most inclusive sense possible. Thus, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise. Structures described herein are to be understood also to refer to functional equivalents of such structures. Language that may be construed to express approximation should be so understood unless the context clearly dictates otherwise.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “having” should be interpreted as “having at least”; the term “includes” should be interpreted as “includes but is not limited to”; the term “example” is used to provide exemplary instances of the item in discussion, not an exhaustive or limiting list thereof; and use of terms like “preferably,” “preferred,” “desired,” “desirable,” or “exemplary” and words of similar meaning should not be understood as implying that certain features are critical, essential, or even important to the structure or function of the invention, but instead as merely intended to highlight alternative or additional features that may or may not be utilized in a particular embodiment of the invention.

Those skilled in the art will also understand that if a specific number of an introduced claim recitation is intended, such an intent will be explicitly recited in the claim, and in the absence of such recitation no such intent is present. For example, as an aid to understanding, the appended claims may contain usage of the introductory phrases “at least one” and “one or more” to introduce claim recitations; however, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.).

All numbers expressing dimensions, quantities of ingredients, reaction conditions, and so forth used in the specification are to be understood as being modified in all instances by the term “about” unless expressly stated otherwise. Accordingly, unless indicated to the contrary, the numerical parameters set forth herein are approximations that may vary depending upon the desired properties sought to be obtained.

The invention provides a composition for improving brain health and cognitive function. By improving cognitive function and brain health, the compositions described herein are also effective for preventing dementia-related diseases and conditions such as Alzheimer's disease. The invention also provides a composition for treating dementia-related diseases and conditions. The invention also provides a composition for treating symptoms of and improving cognitive function in persons with traumatic brain injuries. The invention also provides a composition for providing brain energy to young adults, persons engaged in exercise, and for neonatal uses. Most embodiments of the invention include the following essential ingredients: a source of choline, a source of magnesium, at least one taurine-containing composition, a source of ashwagandha root, and phosphatidylserine. However, embodiments intended for neonatal use by pregnant women should not include the source of ashwagandha root.

The source of choline is selected from among alpha-glycerylphosphorylcholine, citicoline, phosphatidylcholine, phosphocholine, free choline, or a combination of two or more of the foregoing. Among those, alpha-glycerylphosphorylcholine (also known as choline alfoscerate, sn-glycedro-3-phosphocholine, and alpha-GPC) and citicoline are the two most preferred sources of choline.

A source of choline (e.g., alpha-glycerylphosphorylcholine or citicoline) is included in the composition because it promotes the release of acetyl choline in and to the brain. In this composition, the source of choline is also included for its effects in maintaining neurochemical balance in the brain, improving memory and learning ability, promoting cellular health and supporting cellular membranes, and enhancing cognitive functions and preventing cognitive decline. The source of choline also provides further benefits to the composition by increasing muscle mass, endurance performance, and growth hormone secretion; reducing body fat; and preventing reductions in choline levels as a result of exercise. Due to its aforementioned effects, the source of choline serves important functions in the composition by protecting the brain from damage caused by Alzheimer's disease and other age-related disorders.

The source of magnesium is selected from among magnesium glycinate, magnesium L-threonate, magnesium citrate, magnesium oxide, magnesium chloride, magnesium malate, magnesium sulfate, magnesium taurate, magnesium lactate, or a combination of two or more of the foregoing. Magnesium L-threonate is the most preferred source of magnesium with magnesium taurate and magnesium glycinate being the next most preferred alternatives.

The source of magnesium (e.g., magnesium-L-threonate) is included in the composition as an electrolyte and for its effects in enhancing sleep quality, improving synaptic function and density, and reducing stress. The source of magnesium is also included in the composition because it is believed to be effective in reversing age-related cognitive decline. The source of magnesium is also included in the composition for dietary supplemental purposes because it facilitates adenosine triphosphate (ATP) production, acts as an enzymatic co-factor in numerous metabolic processes in the body, and supports cognitive health and ideal brain function. As a source of dietary magnesium, the source of magnesium is also included for its behavioral health effects including its effects in improving focus, concentration, and mood, and raising energy levels. Consumption of appropriate amounts of a source of magnesium such as magnesium-L-threonate may also help to alleviate symptoms related to anxiety, depression, and post-traumatic stress disorder (PTSD).

The at least one taurine-containing composition is selected from among taurine, taurine glycinate, taurine oleate, taurine ethanoleate, taurine ethanolamine, a taurine ester, a taurine salt, a taurine amino alcohol, taurocholic acid, a taurolipid, a lipophilic taurine analog, N-acetyltaurine, N-propionyltaurine, a taurine-fatty acid conjugate, a taurine amide derivative, or a combination of two or more of the foregoing.

The source of ashwagandha root is ashwagandha root powder in exemplary embodiments, but in other embodiment, an extract of ashwagandha may be used. The source of ashwagandha root is included in the composition for its effectiveness in reducing stress, anxiety, depression, and inflammation. Ashwagandha also improves brain function including the speed of information processing, attention, and memory.

Phosphatidylserine, which is a phospholipid found in high concentrations in brain cell membranes, is included in the composition to support memory, learning, and cognitive function. Phosphatidylserine may protect brain cells, particularly the cell membranes, and improve communication between neurons.

### Taurine-Containing Compositions

Taurine-containing and taurine-derived compositions are included in the composition for purposes of promoting health by increasing or promoting uptake or assimilation of nutrients contained by ingestible items. Examples of such taurine-containing compositions suitable for inclusion in the compositions described herein include taurine, taurine glycinate, taurine oleate, taurine ethanoleate, taurine ethanolamine, a taurine ester, a taurine salt, a taurine amino alcohol, taurocholic acid, a taurolipid, a lipophilic taurine analog, N-acetyltaurine, N-propionyltaurine, a taurine-fatty acid conjugate, a taurine amide derivative, or a combination of two or more of the foregoing. The compositions contain or are derived from taurine (or tauric acid) or taurine-containing compounds (such as taurine analogs or taurine derivatives) and are useful as a treatment that can be applied to an ingestible item to control the timing of digestion and to increase shelf-life of the ingestible item. For convenience and without limiting the scope of the foregoing description, as used herein, the term “taurine-containing composition” refers to compositions of the invention that are or include taurine, one or more compounds that contain taurine, or one or more taurine derivatives. Examples of taurine-containing compositions that are suitable for inclusion in the compositions described herein are found elsewhere herein and in U.S. Pat. No. 10,881,125, which is incorporated by reference in its entirety. The ingestible item can be a food, a nutraceutical composition, a nutritional supplement (also called a dietary supplement herein; and such as those described herein), or a pharmaceutical composition. In some embodiments, the taurine-containing composition is a single food-safe compound or composition that is, includes, or is derived from taurine. In other embodiments, the taurine-containing composition includes two or more food-safe compounds or compositions that are, include, or are derived from taurine. The taurine-containing composition can be a taurine ester, a taurine salt, a taurine amino alcohol, or a combination of two or more of the foregoing. For example, the taurine-containing composition can be a taurine analog such as taurine glycinate (also known as glyceryl taurinate), taurine oleate (which is an estolide), taurine ethanoleate, taurine ethanolamine, or a combination of two or more of the foregoing. These taurine analogs are amphiphilic and water soluble. Taurine and its analogs, such as the taurine-containing compositions above, promote the bioavailability of lipid soluble vitamins (e.g., vitamins A, D, E, K, and F) by forming various hydrolysable, water soluble complexes that improve assimilation of the lipid soluble vitamins during digestion. In this way, the taurine-containing compositions produce a “gastroretentive” effect when consumed by delaying or slowing digestion of the ingestible item to extend the release of nutrients the ingestible item contains, thereby improving assimilation of the nutrients. Thus, the taurine-containing compositions described herein do not merely physically protect ingestible items from digestion until more nutrients can be released, but rather, these taurine-containing compositions are active compounds that interact with nutrients like the lipid soluble vitamins to form complexes that are more readily assimilated by a human or other animal's body during digestion.

Because the taurine analogs of the taurine-containing compositions are amphiphilic, they are useful for suspension and colloidal dispersion of lipids in aqueous solutions because they have the ability to solubilize lipids in micelles. When the taurine-containing compositions solubilize lipids in micelles, the lipid tail of the lipid faces inward within each micelle and the acid end of the lipid interacts with the surrounding aqueous solution.

The taurine-containing compositions can form lipid matrixes with polymers that have the ability to “swell” or expand when exposed to acidic conditions (such as in the human digestive tract), which provides a mechanism for sustained release of vitamins and other nutrients as well as pharmaceutical compounds during digestion from consumed ingestible items to which one of the taurine-containing compositions was applied prior to consumption.

The taurine-containing composition is applied to the ingestible item for treatment before the ingestible item is consumed by a human or other animal. The ingestible item may be processed before application of the composition to it. For example, the ingestible item may be cut, diced, chopped, shredded, crushed, blended, mixed, powderized, atomized, reduced to particle or particulate size, placed in suspension, dispersed in a colloid, dissolved in a solution, liquefied, torn into pieces, cooked, heated, cooled, chilled, frozen, or otherwise processed so as to cause some physical and/or chemical change to the ingestible item. The ingestible item may be combined with other ingestible items before or after it is processed and such combination can occur before or after application of the taurine-containing composition. In exemplary embodiments, the taurine-containing composition is applied to a processed ingestible item that has been processed in one or more of the processing manners described above. In the most preferred embodiments, the ingestible item is reduced to particle size and then placed in suspension or in a colloid with the taurine-containing composition. For example, ingestible item that is reduced to particle size can be one or more raw fruits, one or more raw vegetables, or one or more raw fruits plus one or more raw vegetables, which are added to (or to which are added) the taurine-containing composition. In other embodiments, the taurine-containing composition may be applied to a raw ingestible item, i.e., one that has not been processed in any of the processing manners described above.

When the taurine-containing composition is applied to a processed ingestible item that is in suspension or in a colloid (e.g. food particles in suspension or food particles in a colloid), the suspended particles become part of micelles that are formed by the ingestible item being reduced to particle size and placed in (or made part of) the suspension or colloid. The micelles formed by the combination of the taurine-containing composition and the ingestible item placed in suspension or in a colloid allow for a controlled or sustained release of vitamins, minerals, or other nutrients or of pharmaceutical compounds contained by the particles of the ingestible item in the micelles.

In one embodiment, the taurine-containing composition is taurine glycinate only and does not contain any other ingredients.

In another embodiment, the taurine-containing composition is taurine glycinate and also contains one or more other taurine-containing compounds, e.g., one or more of the taurine analogs described herein. In such embodiments, the composition may contain taurine glycinate and one or more other taurine-containing compounds plus one or more other ingredients that do not contain taurine. Examples of such ingredients that do not contain taurine include the other compounds disclosed herein. In alternate embodiments, the composition may contain taurine glycinate and one or more other taurine-containing compounds but excludes any other ingredients that do not contain taurine.

In another embodiment, the taurine-containing composition is taurine glycinate and also contains one or more other ingredients that do not contain taurine but excludes any other taurine-containing compounds. Examples of such ingredients that do not contain taurine include the other compounds disclosed herein.

In one embodiment, the taurine-containing composition is taurine oleate only and does not contain any other ingredients.

In another embodiment, the taurine-containing composition is taurine oleate and also contains one or more other taurine-containing compounds, e.g., one or more of the taurine analogs described herein. In such embodiments, the composition may contain taurine oleate and one or more other taurine-containing compounds plus one or more other ingredients that do not contain taurine. Examples of such ingredients that do not contain taurine include the other compounds disclosed herein. In alternate embodiments, the composition may contain taurine oleate and one or more other taurine-containing compounds but excludes any other ingredients that do not contain taurine.

In another embodiment, the taurine-containing composition is taurine oleate and also contains one or more other ingredients that do not contain taurine but excludes any other taurine-containing compounds. Examples of such ingredients that do not contain taurine include the other compounds disclosed herein.

In one embodiment, the taurine-containing composition is taurine ethanoleate only and does not contain any other ingredients.

In another embodiment, the taurine-containing composition is taurine ethanoleate and also contains one or more other taurine-containing compounds, e.g., one or more of the taurine analogs described herein. In such embodiments, the composition may contain taurine ethanoleate and one or more other taurine-containing compounds plus one or more other ingredients that do not contain taurine. Examples of such ingredients that do not contain taurine include the other compounds disclosed herein. In alternate embodiments, the composition may contain taurine ethanoleate and one or more other taurine-containing compounds but excludes any other ingredients that do not contain taurine.

In another embodiment, the taurine-containing composition is taurine ethanoleate and also contains one or more other ingredients that do not contain taurine but excludes any other taurine-containing compounds. Examples of such ingredients that do not contain taurine include the other compounds disclosed herein.

In one embodiment, the taurine-containing composition is taurine ethanolamine only and does not contain any other ingredients.

In another embodiment, the taurine-containing composition is taurine ethanolamine and also contains one or more other taurine-containing compounds, e.g., one or more of the taurine analogs described herein. In such embodiments, the composition may contain taurine ethanolamine and one or more other taurine-containing compounds plus one or more other ingredients that do not contain taurine. Examples of such ingredients that do not contain taurine include the other compounds disclosed herein. In alternate embodiments, the composition may contain taurine ethanolamine and one or more other taurine-containing compounds but excludes any other ingredients that do not contain taurine.

In another embodiment, the taurine-containing composition is taurine ethanolamine and also contains one or more other ingredients that do not contain taurine but excludes any other taurine-containing compounds. Examples of such ingredients that do not contain taurine include the other compounds disclosed herein.

In one embodiment, the taurine-containing composition contains taurine glycinate and taurine oleate only. In another embodiment, the taurine-containing composition contains only taurine glycinate, taurine oleate, and one or more other taurine-containing compounds. In yet another embodiment, the taurine-containing composition contains taurine glycinate, taurine oleate, one or more other taurine-containing compounds, and one or more ingredients that do not contain taurine. In still another embodiment, the taurine-containing composition contains only taurine glycinate, taurine oleate, and one or more other compounds that do not contain taurine. Examples of such ingredients that do not contain taurine include the other compounds disclosed herein.

In one embodiment, the taurine-containing composition contains taurine glycinate and taurine ethanoleate only. In another embodiment, the taurine-containing composition contains only taurine glycinate, taurine ethanoleate, and one or more other taurine-containing compounds. In yet another embodiment, the taurine-containing composition contains taurine glycinate, taurine ethanoleate, one or more other taurine-containing compounds, and one or more ingredients that do not contain taurine. In still another embodiment, the taurine-containing composition contains only taurine glycinate, taurine ethanoleate, and one or more other compounds that do not contain taurine. Examples of such ingredients that do not contain taurine include the other compounds disclosed herein.

In one embodiment, the taurine-containing composition contains taurine glycinate and taurine ethanolamine only. In another embodiment, the taurine-containing composition contains only taurine glycinate, taurine ethanolamine, and one or more other taurine-containing compounds. In yet another embodiment, the taurine-containing composition contains taurine glycinate, taurine ethanolamine, one or more other taurine-containing compounds, and one or more ingredients that do not contain taurine. In still another embodiment, the taurine-containing composition contains only taurine glycinate, taurine ethanolamine, and one or more other compounds that do not contain taurine. Examples of such ingredients that do not contain taurine include the other compounds disclosed herein.

In one embodiment, the taurine-containing composition contains taurine oleate and taurine ethanoleate only. In another embodiment, the taurine-containing composition contains only taurine oleate, taurine ethanoleate, and one or more other taurine-containing compounds. In yet another embodiment, the taurine-containing composition contains taurine oleate, taurine ethanoleate, one or more other taurine-containing compounds, and one or more ingredients that do not contain taurine. In still another embodiment, the taurine-containing composition contains only taurine oleate, taurine ethanoleate, and one or more other compounds that do not contain taurine. Examples of such ingredients that do not contain taurine include the other compounds disclosed herein.

In one embodiment, the taurine-containing composition contains taurine oleate and taurine ethanolamine only. In another embodiment, the taurine-containing composition contains only taurine oleate, taurine ethanolamine, and one or more other taurine-containing compounds. In yet another embodiment, the taurine-containing composition contains taurine oleate, taurine ethanolamine, one or more other taurine-containing compounds, and one or more ingredients that do not contain taurine. In still another embodiment, the taurine-containing composition contains only taurine oleate, taurine ethanolamine, and one or more other compounds that do not contain taurine. Examples of such ingredients that do not contain taurine include the other compounds disclosed herein.

In one embodiment, the taurine-containing composition contains taurine ethanoleate and taurine ethanolamine only. In another embodiment, the taurine-containing composition contains only taurine ethanoleate, taurine ethanolamine, and one or more other taurine-containing compounds. In yet another embodiment, the taurine-containing composition contains taurine ethanoleate, taurine ethanolamine, one or more other taurine-containing compounds, and one or more ingredients that do not contain taurine. In still another embodiment, the taurine-containing composition contains only taurine ethanoleate, taurine ethanolamine, and one or more other compounds that do not contain taurine. Examples of such ingredients that do not contain taurine include the other compounds disclosed herein.

In one embodiment, the taurine-containing composition contains taurine glycinate, taurine oleate, and taurine ethanoleate only. In another embodiment, the taurine-containing composition contains only taurine glycinate, taurine oleate, taurine ethanoleate, and one or more other taurine-containing compounds. In yet another embodiment, the taurine-containing composition contains taurine glycinate, taurine oleate, taurine ethanoleate, one or more other taurine-containing compounds, and one or more ingredients that do not contain taurine. In still another embodiment, the taurine-containing composition contains only taurine glycinate, taurine oleate, taurine ethanoleate, and one or more other compounds that do not contain taurine. Examples of such ingredients that do not contain taurine include the other compounds disclosed herein.

In one embodiment, the taurine-containing composition contains taurine glycinate, taurine oleate, and taurine ethanolamine only. In another embodiment, the taurine-containing composition contains only taurine glycinate, taurine oleate, taurine ethanolamine, and one or more other taurine-containing compounds. In yet another embodiment, the taurine-containing composition contains taurine glycinate, taurine oleate, taurine ethanolamine, one or more other taurine-containing compounds, and one or more ingredients that do not contain taurine. In still another embodiment, the taurine-containing composition contains only taurine glycinate, taurine oleate, taurine ethanolamine, and one or more other compounds that do not contain taurine. Examples of such ingredients that do not contain taurine include the other compounds disclosed herein.

In one embodiment, the taurine-containing composition contains taurine glycinate, taurine ethanoleate, and taurine ethanolamine only. In another embodiment, the taurine-containing composition contains only taurine glycinate, taurine ethanoleate, taurine ethanolamine, and one or more other taurine-containing compounds. In yet another embodiment, the taurine-containing composition contains taurine glycinate, taurine ethanoleate, taurine ethanolamine, one or more other taurine-containing compounds, and one or more ingredients that do not contain taurine. In still another embodiment, the taurine-containing composition contains only taurine glycinate, taurine ethanoleate, taurine ethanolamine, and one or more other compounds that do not contain taurine. Examples of such ingredients that do not contain taurine include the other compounds disclosed herein.

In one embodiment, the taurine-containing composition contains taurine oleate, taurine ethanoleate, and taurine ethanolamine only. In another embodiment, the taurine-containing composition contains only taurine oleate, taurine ethanoleate, taurine ethanolamine, and one or more other taurine-containing compounds. In yet another embodiment, the taurine-containing composition contains taurine oleate, taurine ethanoleate, taurine ethanolamine, one or more other taurine-containing compounds, and one or more ingredients that do not contain taurine. In still another embodiment, the taurine-containing composition contains only taurine oleate, taurine ethanoleate, taurine ethanolamine, and one or more other compounds that do not contain taurine. Examples of such ingredients that do not contain taurine include the other compounds disclosed herein.

In the foregoing embodiments, where an embodiment is described as including “only” certain specific, identified ingredients, such embodiment of the taurine-containing composition excludes any other ingredients not identified.

In addition to one or more of the taurine analogs above, in some embodiments, the composition also includes one or more fatty acids such as those described elsewhere herein. In exemplary embodiments containing fatty acids, the fatty acids can be 12-20 carbon molecule fatty acids.

Some embodiments of the composition include a source of vitamin B5. The source of vitamin B5 is selected from among D-pantothenic acid, calcium pantothenate, sodium pantothenate, pantethine, or a combination of two or more of the foregoing. Pantethine is the most preferred source of vitamin B5.

Oleic acid is included in some embodiments of the composition for its effects in improving and maintaining cognitive and memory functions. Some studies have shown that dietary intake of oleic acid is related to a reduction in the risk of dementia-related mortality.

Oleanolic acid is included in some embodiments of the composition for its effects in inhibiting cellular inflammatory processes.

Ursolic acid is included in some embodiments of the composition for its effects in preventing cognitive declines. Studies conducted using mice have shown that dietary intake of ursolic acid in effective amounts can treat high fat diet-induced cognitive impairments by blocking endoplasmic reticulum stress and IκB kinase β/nuclear factor-κB-mediated inflammatory pathways, reduces domoic acid-induced cognitive deficits, improves cognition deficits and reduces oxidative damage in the brain of senescent mice induced by D-galactose, and reduces lipopolysaccharide-induced cognitive deficits by suppressing p38/NF-κB mediated inflammatory pathways.

Curcumin is included in some embodiments of the composition to serve as an anti-oxidant and for its anti-inflammatory effects. Curcumin may reduce brain inflammation and oxidative stress. Curcumin may also inhibit amyloid-beta aggregation. Turmeric is a source of curcumin and can be included in the composition in the form of a powder (e.g., a root powder) or as an extract.

Piperine is included in some embodiments of the composition to enhance nutrient absorption, decrease inflammation, and boost brain functions. Piperine acts as a bioavailability enhancer and is particularly useful in enhancing the absorption of certain compounds including curcumin.

Quercetin dihydrate is included in some embodiments of the composition as an anti-inflammatory agent and an antioxidant. Quercetin dihydrate scavenges free radicals and reduces neuroinflammation, which provides a neuroprotection benefit. Quercetin dihydrate may also inhibit β-amyloid plaque formation, which is one indicator of Alzheimer's disease.

Alpha-lipoic acid (ALA) is included in some embodiments of the composition to provide mitochondrial support and as an antioxidant. ALA is believed to preserve cognitive function and also assists in reducing oxidative stress and helps maintain cellular energy metabolism. In some studies, ALA has also been shown to slow cognitive decline in mild cases of Alzheimer's disease.

Nicotinamide, which is a form of vitamin B3 and is a precursor to NAD+, is included in some embodiments of the composition to enhance cellular energy and DNA repair. Nicotinamide supports mitochondrial function and neuronal health and is believed to improve cognitive function and reduce tau pathologies, which are a type of neurogenerative diseases.

Astaxanthin is included in some embodiments of the composition as an antioxidant, which protects neurons from oxidative damage and also supports mitochondrial health. Some studies show that astaxanthin has neuroprotective effects.

Glutamine is included in some embodiments of the composition to provide energy to the brain and for its role as a neurotransmitter precursor for glutamate and GABA, both of which have important roles in synaptic function. Glutamine is believed to support and improve cognitive health.

D-ribose is included in some embodiments of the composition to promote cellular energy metabolism by supporting ATP synthesis. These processes are believed to improve cellular energy deficits that are often observed in neurodegeneration. D-ribose is believed to provide a benefit by reducing mitochondrial dysfunction.

In addition to the aforementioned ingredients, the composition may further include at least one of galantamine, huperzine-A, vitamin B5, L-tyrosine, and beta-hydroxybutyrate.

Galantamine, which is an acetylcholinesterase inhibitor, is included in some embodiments of the composition for its effects in treating and slowing early-stage cognitive decline related to Alzheimer's disease, age-related dementia, and memory impairments. Galantamine increases the amount of acetylcholine (ACh), a neurotransmitter, in the brain by inhibiting enzymatic breakdown of acetylcholine by acetylcholinesterase, thereby increasing and maintaining acetylcholine levels to enhance certain cognitive functions including memory processing, reasoning, and thinking.

Huperzine-A is included in some embodiments of the composition for its effects in inhibiting the breakdown of acetylcholine by acetylcholinesterase. Like galantamine, huperzine-A is an acetylcholinesterase inhibitor and a N-Methyl-D-aspartate (NMDA) receptor antagonist, which is capable of passing through the blood-brain barrier.

Vitamin B5 is included in some embodiments (and particularly in exemplary embodiments) of the composition for its effects in synthesizing acetylcholine. Vitamin B5 deficiency in humans causes disruptions in the body's natural production of acetylcholine, which can cause negative neurological symptoms. Dietary intake of vitamin B5 enhances the body's production of acetylcholine.

L-tyrosine is included in some embodiments of the composition for its effects in improving cognitive function in patients with certain types of dementia such as, for example, Lewy body dementia. L-tyrosine is converted to levodopa (also known as L-dopa) in the brain. Levodopa is a precursor to the synthesis of dopamine, which is a neurotransmitter. Higher baseline serum concentrations of tyrosine have been linked to better cognitive performance in patients with Lewy body dementia.

Beta-hydroxybutyrate (also known as β-hydroxybutyrate, or BHB) has several benefits, particularly for younger users and athletic adults. First, BHB can be used as an energy source by the brain when blood glucose is low. Second, BHB is able to cross the blood-brain barrier into the central nervous system during increased exercise. Third, high levels of BHB improves cognition in patients with Alzheimer's disease. Beta hydroxybutyrate is included in some embodiments of the composition for its aforementioned effects.

Prolonged exercise increases plasma BHB concentrations, which induces promoters of the brain-derived neurotrophic factor (BDNF) gene, which is believed to have clinical relevance in the treatment of cognitive impairment related to Alzheimer's disease and other age-related dementia diseases and conditions. Further, BHB inhibits the NLRP3 inflammasome, which is believed to be related to neuroinflammation and the progression of Alzheimer's disease.

The following exemplary embodiments of the composition include the following ingredients and the mass for each ingredient in the daily dose:

### Composition 1

oleic acid
oleanolic acid
ursolic acid
curcumin
piperine
alpha-glycerylphosphorylcholine
magnesium-L-threonate
at least one taurine-containing composition
ashwagandha root
phosphatidylserine

### Composition 2

oleic acid
oleanolic acid
ursolic acid
galantamine
huperzine-A
vitamin B5

### Composition 3

curcumin
piperine
alpha-glycerylphosphorylcholine
magnesium-L-threonate
at least one taurine-containing composition
ashwagandha root
phosphatidylserine

### Composition 4

about 250 to about 1,000 mg of citicoline, or about 300 to about 600 mg of alpha-glycerylphosphorylcholine
about 1,000 to about 2,000 mg of magnesium L-threonate, or about 100 to about 300 mg of magnesium glycinate
about 300 to about 600 mg of a source of ashwagandha root
about 100 to about 300 mg of phosphatidylserine
about 5 to about 15 mg of piperine
about 250 to about 1,000 mg of at least one taurine-containing composition
about 250 to about 700 mg of curcumin
about 100 to about 200 mg of L-theanine
about 50 to about 200 mg of ursolic acid
about 1,500 to about 3,000 mg of acetyl-L-carnitine
about 250 to about 1,000 mg of a source of vitamin B5

### Composition 5

about 250 mg of citicoline, or about 300 mg of alpha-glycerylphosphorylcholine
about 1,000 mg of magnesium L-threonate, or about 100 to about 150 mg of magnesium glycinate
about 300 mg of a source of ashwagandha root
about 100 mg of phosphatidylserine
about 5 mg of piperine
about 250 mg of at least one taurine-containing composition
about 250 mg of curcumin
about 100 mg of L-theanine
about 50 to about 75 mg of ursolic acid
about 1,500 mg of acetyl-L-carnitine
about 250 mg of a source of vitamin B5

### Composition 6

about 500 mg of citicoline, or about 400 to about 500 mg of alpha-glycerylphosphorylcholine
about 1,500 mg of magnesium L-threonate, or about 200 mg of magnesium glycinate
about 500 mg of a source of ashwagandha root
about 200 mg of phosphatidylserine
about 10 mg of piperine
about 500 mg of at least one taurine-containing composition
about 500 mg of curcumin
about 150 mg of L-theanine
about 100 mg of ursolic acid
about 1,500 to about 2,000 mg of acetyl-L-carnitine
about 500 mg of a source of vitamin B5

### Composition 7

about 500 to about 1,000 mg of citicoline, or about 600 mg of alpha-glycerylphosphorylcholine
about 2,000 mg of magnesium L-threonate, or about 250 to about 300 mg of magnesium glycinate
about 600 mg of a source of ashwagandha root
about 300 mg of phosphatidylserine
about 10 to about 15 mg of piperine
about 1,000 mg of at least one taurine-containing composition
about 500 to about 700 mg of curcumin
about 200 mg of L-theanine
about 150 to about 200 mg of ursolic acid
about 2,000 to about 3,000 mg of acetyl-L-carnitine
about 1,000 mg of a source of vitamin B5

The following exemplary embodiments of the composition include the following ingredients and the mass for each ingredient in the daily dose. The masses provided for these embodiments are “not less than” the amounts indicated:

### Composition 8

about 180 mg of turmeric root powder
about 12 mg of piperine
about 220 mg of alpha-glycerylphosphorylcholine
about 450 mg of magnesium glycinate
about 350 mg of a taurine-containing composition
about 70 mg of ashwagandha root powder
about 200 mg of phosphatidylserine
about 60 mg olive leaf and fruit extract (for oleic acid, oleanolic acid, and ursolic acid)
about 10 mg of huperzine A
about 20 mg of a source of vitamin B5

Composition 8 is an exemplary embodiment useful for treating the symptoms of Alzheimer's disease.

### Composition 9

about 100 mg of turmeric root powder
about 9 mg of piperine
about 150 mg of alpha-glycerylphosphorylcholine
about 350 mg of magnesium glycinate
about 200 mg of a taurine-containing composition
about 70 mg of ashwagandha root powder
about 150 mg of phosphatidylserine
about 60 mg olive leaf and fruit extract (for oleic acid, oleanolic acid, and ursolic acid)
about 10 mg of huperzine A
about 20 mg of a source of vitamin B5

Composition 9 is an exemplary embodiment useful for preventing Alzheimer's disease and the symptoms thereof.

### Composition 10

about 300 mg of magnesium glycinate
about 150 mg of alpha-glycerylphosphorylcholine
about 50 mg of a taurine-containing composition
about 4 mg of piperine
about 75 mg of turmeric root powder
about 90 mg of phosphatidylserine

Composition 10 is an exemplary neonatal embodiment useful for preventing and treating Alzheimer's disease and the symptoms thereof, which is safe for use during pregnancy.

### Composition 11

about 100 mg of turmeric root powder
about 9 mg of piperine
about 150 mg of alpha-glycerylphosphorylcholine
about 350 mg of magnesium glycinate
about 200 mg of a taurine-containing composition
about 70 mg of ashwagandha root powder
about 150 mg of phosphatidylserine
about 60 mg olive leaf and fruit extract (for oleic acid, oleanolic acid, and ursolic acid)
about 10 mg of huperzine A
about 20 mg of a source of vitamin B5
about 80 mg of L-tyrosine
about 80 mg of beta-hydroxybutyrate

Composition 11 is an exemplary embodiment useful for providing a source of energy for the brain during exercise and for preventing Alzheimer's disease and the symptoms thereof.

### Composition 12

about 60 mg of turmeric root powder
about 5 mg of piperine
about 80 mg of alpha-glycerylphosphorylcholine
about 200 mg of magnesium glycinate
about 80 mg of a taurine-containing composition
about 40 mg of ashwagandha root powder
about 80 mg of phosphatidylserine
about 60 mg olive leaf and fruit extract (for oleic acid, oleanolic acid, and ursolic acid)
about 10 mg of huperzine A
about 20 mg of a source of vitamin B5
about 80 mg of L-tyrosine
about 40 mg of beta-hydroxybutyrate

Composition 12 is an exemplary embodiment useful for preventing dementia and the symptoms thereof.

### Composition 13

about 90 mg of turmeric root powder
about 10 mg of piperine
about 120 mg of alpha-glycerylphosphorylcholine
about 300 mg of magnesium glycinate
about 130 mg of a taurine-containing composition
about 70 mg of ashwagandha root powder
about 110 mg of phosphatidylserine
about 60 mg olive leaf and fruit extract (for oleic acid, oleanolic acid, and ursolic acid)
about 10 mg of huperzine A
about 20 mg of a source of vitamin B5
about 60 mg of beta-hydroxybutyrate

Composition 13 is an exemplary embodiment useful for treating dementia and the symptoms thereof.

### Composition 14

about 100 mg of quercetin dihydrate
about 100 mg turmeric root powder
about 300 mg of alpha-lipoic acid
about 200 mg of magnesium glycinate
about 250 mg of nicinamide
about 500 mg of a taurine-containing composition
about 10 mg of piperine
about 50 mg phosphatidylserine
about 100 mg of phosphatidylcholine
about 10 mg of astaxanthin
about 100 mg of glutamine
about 100 mg of D-ribose

Composition 14 is an exemplary embodiment useful for improving cognitive function and treating the symptoms of persons with traumatic brain injuries.

The foregoing compositions and any other embodiments contemplated herein may also include one or more of the following additional ingredients: citric acid, a flavoring agent; gum acacia, a composition for masking or reducing bitter flavors, a sweetener, and bamboo extract.

The foregoing compositions can be blended into various compositions having different amounts, percentages by weight, or ranges of masses, weights, or volumes for each ingredient. Such blends may be optimized based on the intended purposes for consumption, e.g., prevention of age-related dementia diseases and conditions, treatment of age-related dementia diseases and conditions, brain energy source for young adults and persons engaged in exercise, or neonatal uses. Embodiments used for neonatal purposes may not include ashwagandha root.

One exemplary embodiment of the composition includes oleic acid, oleanolic acid, ursolic acid, curcumin, piperine, alpha-GPC, magnesium-L-threonate, at least one taurine-containing composition, ashwagandha root, and phosphatidylserine.

Another exemplary embodiment of the composition includes oleic acid, oleanolic acid, ursolic acid, curcumin, piperine, alpha-GPC, magnesium-L-threonate, at least one taurine-containing composition, ashwagandha root, phosphatidylserine, galantamine, huperzine-A, and vitamin B5.

Another exemplary embodiment of the composition includes oleic acid, oleanolic acid, ursolic acid, curcumin, piperine, alpha-GPC, magnesium-L-threonate, at least one taurine-containing composition, ashwagandha root, phosphatidylserine, and vitamin B5.

Another exemplary embodiment of the composition includes oleic acid, oleanolic acid, ursolic acid, curcumin, piperine, alpha-GPC, magnesium-L-threonate, at least one taurine-containing composition, ashwagandha root, phosphatidylserine, galantamine, and vitamin B5.

Another exemplary embodiment of the composition includes oleic acid, oleanolic acid, ursolic acid, curcumin, piperine, alpha-GPC, magnesium-L-threonate, at least one taurine-containing composition, ashwagandha root, phosphatidylserine, huperzine-A, and vitamin B5.

One exemplary embodiment of the composition includes oleic acid, oleanolic acid, ursolic acid, curcumin, piperine, alpha-GPC, magnesium-L-threonate, at least one taurine-containing composition, ashwagandha root, phosphatidylserine, galantamine, huperzine-A, vitamin B5, tyrosine, and beta hydroxybutyrate.

An exemplary embodiment of the composition for neonatal uses includes oleic acid, oleanolic acid, ursolic acid, curcumin, piperine, alpha-GPC, magnesium-L-threonate, at least one taurine-containing composition, phosphatidylserine, galantamine, huperzine-A, and vitamin B5.

The composition may be produced and provided in the form of a pill, caplet, tablet, liquid, powder, or gel for human consumption. In one embodiment, the composition may be manufactured pre-mixed as part of a food or beverage. For example, in one embodiment, the composition may be mixed by a consumer into, or pre-mixed with, a salad dressing to be eaten with a salad or other meal. In exemplary embodiments, the composition is provided in the form of a powdered drink mix.

Among other reasons, the compositions described herein are believed to be effective because they reduce levels of homocysteine in the body. Elevated homocysteine levels are associated with calcified plaques in the vascular system. Increases in a patient's homocysteine level are linked to increases in the prevalence and progression of vascular calcification, which is an indicator of atherosclerosis. Higher than normal homocysteine levels are believed to be a risk factor not only for heart disease but also for cerebrovascular diseases and conditions. The composition's ability to lower homocysteine levels is believed to be one factor in its efficacy in improving cognitive function and brain health and in preventing and treating age-related dementia diseases and conditions.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition for improving cognitive function and for preventing and treating symptoms of Alzheimer's disease and dementia, the composition comprising:
a source of choline;
a source of magnesium;
at least one taurine-containing composition;
a source of ashwagandha root; and
phosphatidylserine.

2. The composition of claim 1, wherein the source of choline comprises alpha-glycerylphosphorylcholine or citicoline.

3. The composition of claim 1, wherein the source of magnesium comprises magnesium L-threonate or magnesium glycinate.

4. The composition of claim 1, further comprising a source of vitamin B5 comprising D-pantothenic acid, calcium pantothenate, sodium pantothenate, pantethine, or a combination of two or more of the foregoing.

5. The composition of claim 1, further comprising:
a source of turmeric root;
piperine;
uridine monophosphate;
L-theanine; and
vitamin B5.

6. The composition of claim 1, further comprising:
a source of turmeric root;
piperine;
oleic acid;
oleanolic acid;
ursolic acid;
huperzine A; and
vitamin B5.

7. The composition of claim 1, further comprising:
a source of turmeric root; and
piperine.

8. The composition of claim 1, further comprising:
a source of turmeric root;
piperine;
oleic acid;
oleanolic acid;
ursolic acid;
huperzine A;
L-tyrosine;
beta-hydroxybutyrate; and
vitamin B5.

9. The composition of claim 1, further comprising:
quercetin dihydrate;
a source of turmeric root;
alpha-lipoic acid;
nicinamide;
piperine;
astaxanthin;
glutamine; and
D-ribose.

10. The composition of claim 1, further comprising:
piperine;
curcumin;
L-theanine;
ursolic acid;
acetyl-L-carnitine; and
vitamin B5.

11. The composition of claim 10, comprising:
about 250 to about 1,000 mg of citicoline, or about 300 to about 600 mg of alpha-glycerylphosphorylcholine;
about 1,000 to about 2,000 mg of magnesium L-threonate, or about 100 to about 300 mg of magnesium glycinate;
about 300 to about 600 mg of a source of ashwagandha root;
about 100 to about 300 mg of phosphatidylserine;
about 5 to about 15 mg of piperine;
about 250 to about 1,000 mg of at least one taurine-containing composition;
about 250 to about 700 mg of curcumin;
about 100 to about 200 mg of L-theanine;
about 50 to about 200 mg of ursolic acid;
about 1,500 to about 3,000 mg of acetyl-L-carnitine; and
about 250 to about 1,000 mg of vitamin B5.

12. The composition of claim 1, wherein the source of choline comprises: alpha-glycerylphosphorylcholine, citicoline, phosphatidylcholine, phosphocholine, free choline, or a combination of two or more of the foregoing.

13. The composition of claim 1, wherein the source of magnesium comprises: magnesium glycinate, magnesium L-threonate, magnesium citrate, magnesium oxide, magnesium chloride, magnesium malate, magnesium sulfate, magnesium taurate, magnesium lactate, or a combination of two or more of the foregoing.

14. The composition of claim 1, wherein the at least one taurine-containing composition comprises: taurine, taurine glycinate, taurine oleate, taurine ethanoleate, taurine ethanolamine, a taurine ester, a taurine salt, a taurine amino alcohol, taurocholic acid, a taurolipid, a lipophilic taurine analog, N-acetyltaurine, N-propionyltaurine, a taurine-fatty acid conjugate, a taurine amide derivative, or a combination of two or more of the foregoing.

15. The composition of claim 1, further comprising at least one of the following additional ingredients: citric acid; a flavoring agent; gum acacia; a composition for masking or reducing bitter flavors; a sweetener, and bamboo extract.

16. A composition for preventing and treating symptoms of Alzheimer's disease and dementia, the composition comprising:
alpha-glycerylphosphorylcholine;
magnesium glycinate;
at least one taurine-containing composition;
a source of ashwagandha root;
phosphatidylserine;
a source of turmeric root;
piperine;
oleic acid;
oleanolic acid;
ursolic acid;
huperzine A; and
vitamin B5.

17. The composition of claim 16, further comprising L-tyrosine, beta-hydroxybutyrate, or both.

18. The composition of claim 16, further comprising at least one of the following additional ingredients: citric acid; a flavoring agent; gum acacia; a composition for masking or reducing bitter flavors; a sweetener; and bamboo extract.

19. A composition for preventing and treating symptoms of Alzheimer's disease and dementia, the composition comprising:
alpha-glycerylphosphorylcholine;
magnesium glycinate;
at least one taurine-containing composition;
phosphatidylserine;
a source of turmeric root; and
piperine.

20. A composition for improving cognitive function and treating symptoms of traumatic brain injury, the composition comprising:
magnesium glycinate;
at least one taurine-containing composition;
a source of ashwagandha root;
phosphatidylserine;
quercetin dihydrate;
a source of turmeric root;
alpha-lipoic acid;
nicinamide;
piperine;
astaxanthin;
glutamine; and
D-ribose.
